(19) United States
(12) Patent Application Publication
(10) Pub. No.: US 2023/0250381 A1
(43) Pub. Date: Aug. 10, 2023

(54) System and Method for Continuous Cell Production

(71) Applicant: National Taiwan University, Taipei City (TW)

(72) Inventors: Tai-Horng Young, Taipei City (TW); Chia-Hsiang Yen, Chiayi County (TW); Ying-Syuan Wu, New Taipei City (TW); Chiao-Chi Shih, Changhua County (TW)

(21) Appl. No.: 18/107,502

(22) Filed: Feb. 9, 2023

(30) Foreign Application Priority Data
Feb. 9, 2022 (TW) 111104802

(51) Int. Cl. C12M 1/12; C12N 5/077; C12N 5/0775; C12N 5/071

(52) U.S. Cl. CPC C12M 25/00; C12N 5/0656; C12N 5/0667; C12N 5/0684; C12N 2539/00; C12N 2533/72; C12N 2533/30

(57) ABSTRACT

The application provides a system for continuous cell production, comprising: a culture container; and a polymer blended layer arranged on the inner surface of the culture container; wherein, the polymer blended layer is a pH-responsive polymer blended with nylon. Additionally, a method for continuous cell production using the system of the present application is provided.

### CROSS-REFERENCE TO RELATED APPLICATIONS

This application claims the priority of patent application Ser. No. 11/104,802 filed on Feb. 9, 2022, in the State Intellectual Property Office of the Republic of China, the disclosure of which is incorporated herein by reference.

### BACKGROUND OF THE INVENTION

### Field of the Invention

The present application relates to a system and a method for continuous cell production using a polymer blended layer, particularly a pH-responsive polymer blended with nylon.

### Descriptions of the Related Art

For more than the past decade, the pH-responsive polymer has been applied in the field of biomaterials, including applications of drug/gene release, tissue engineering, separation procedures, etc. Since the rise of environmental protection awareness, however, developers were not only seeking excellence in performance but also returning to nature. Finding natural materials that can be sustainably reusable while allowing for their performance is expected.

In general speaking, animal cells can be divided into three types, suspension cells (such as blood cells, lymphoid tissue cells, hematopoietic stem cells), adherent cells (such as mesenchymal stem cells, epithelial cells), and the cells between the two types, according to their growth mode. Among them, the adherent cells need to be attached to the object to grow. To separate the cells from the surface of the object, therefore, special treatment is required. Nevertheless, the conventional procedures of the treatment are usually too complicated, or the structure of the cells would be destroyed during the separation process. It results in the death of the cells and will affect the follow-up research.

In this regard, the Patent Publication No. 1432577 of the Republic of China discloses the use of pH-responsive polymers (such as chitosan) as the substrate of the culture plate or coated on the culture plate. The attachment or detachment of cells are controlled by adjusting the pH value of the cell culture medium. The cells can be easily detached without using enzymes or a large amount of washing to avoid cells damage or death.

The Patent Publication No. 1558813 of the Republic of China also discloses the blend of pH-responsive polymers (such as chitosan) with high molecular polymers (such as PCL) as the substrate of the culture dish or coated on the culture dish for cell culture. Besides to control the attachment or detachment of the cells by adjusting the pH value of the cell culture medium, it is also possible to control the efficiency (the amount of cell attachment/detachment) and speed (the rate of cell attachment/detachment) of attachment and detachment according to the blend of different types of polymers and their respective ratios, and further achieve the effect of cell purification or sorting.

Although prior arts have solved the problem of the detachment of cells in cell culture, however, there is a continuous demand for sufficient quantity and good quality of cells in academic research or industrial use. Therefore, it is still necessary to dilute, re-seeding, and re-culturing after harvesting cells, and let the cells re-attach and grow then detach and be harvested again. The operations are repeated to achieve continuous cell harvesting for use. However, the steps are cumbersome, and repeated attachment and detachment are not conducive to cell growth. At the same time, the effective expansion of stem cells during culture and the primary sternness profile are related to the quality of the cells. The re-seeded cells may also fail to attach, be unable to expand or have poor activity, etc. due to the problem of cell quality. Hence, there is still a need for a new cell culture technology that can provide effective and substantial benefits for academic research and industry in terms of cell quantity and quality.

### SUMMARY OF THE INVENTION

In view of the problems of the prior art, the present application provides a system for continuous cell production, comprising:

a culture container, and

a polymer blended layer arranged on the inner surface of the culture container,

In one embodiment, system for continuous cell production further comprise a culture medium and/or a cell.

In one embodiment, the pH-responsive polymer is selected from the group consisting of polymers with amine groups, chitosan, polylysine, polyacrylamine, polyacrylamide, polyhexamethylenediamine, polypropylenediamine, polybutylenediamine, polypentamethylenediamine and combinations thereof.

In one embodiment, the ratio of the blended weight of the pH-responsive polymer and nylon is 1:1 to 10:1.

In one embodiment, the polymer blended layer is prepared by mixing solutions of the pH-responsive polymer and nylon uniformly, coating onto the inner surface of the container, and drying it. In another embodiment, the polymer blended layer is a coating layer of the pH-responsive polymer with a plurality of nylon regions.

Besides, the present application also provides a method for continuous cell production comprising:
(1) providing the system of the present application,
(2) controlling the pH value of the culture medium to the range of 6.9-7.4 and cultivating the cell, and
(3) adjusting the pH value of the culture medium to the range of 7.5-8.5 to detach the cells.

In one embodiment, Step (2) and Step (3) are performed repeatedly. Preferably, Step (2) and Step (3) may be performed repeatedly more than 3 times.

In one embodiment, the cell is selected from the group consisting of stem cell, preferably adipose-derived stem cell, mesenchymal stem cell or skin stem cell; fibroblast cell, preferably foreskin fibroblast or embryonic fibroblast; epithelial cell, preferably proximal renal tubular epithelial cell; cancer cell, preferably epithelial lung cancer cell; keratinocyte; corneal stromal cell and epidermal cell.

Preferably, the polymer blended layer of chitosan-nylon can be used in the present application to build the system for continuous cell production. The attachment and detachment of the cells can be controlled by adjusting the pH value of the medium since the chitosan has pH-responsive properties but is not conducive to cell growth. On the contrary, nylon does not have pH-responsive properties, but it has good properties for cell attachment and growth, which render the cells can still attach when the pH value of the medium is adjusted, and the attached cells can continue to expand when the pH value of the medium returns to control. The method of the present application is to allow cells to attach to the blended material under the control of the appropriate pH value. Then the pH value of the medium is adjusted, chitosan kicks in to detach the cells and the cells are harvested. After that, the pH value of the medium is returned to the control of the initial pH value, and the remaining cells keep attaching to the nylon and continue to grow to the full plate. Next, the same operations are repeated to let the cells detach and be harvested. Such repeated operation is a system and a method for continuous cell production.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 shows the experimental results of cells cultured in systems with different materials.

FIG. 2 shows the cell viability results of cells cultured in the extracted mediums from different materials in different cycles.

FIG. 3 shows the experimental results of cells cultured on different single materials.

FIG. 4 shows the cell viability results of cells cultured on different single materials.

FIG. 5 the experimental results of cell culture with different cells and blending ways.

### DETAILED DESCRIPTION OF THE PREFERRED EMBODIMENT

Reference will be made in detail description to the exemplary embodiments and drawings for being more readily understood to the advantages and features of the present invention, as well as the methods of attaining them. However, the present invention may be carried out in many different forms and should not be construed as limited to the embodiments set forth herein. Conversely, these embodiments are provided to render the present disclosure to be conveyed the scope of the present invention more thoroughly, completely, and fully to one having ordinary skill in the art of the present invention. Moreover, the present invention would be defined only by the appended claims. As used herein, the term “and/or” includes any and all combinations of one or more of the associated listed components.

Unless otherwise defined, all terms (including technical and scientific terms) used herein have the same meaning as generally understood by one having ordinary skill in the art of the present invention. It will be more understandable that, for example, the terms defined in commonly used dictionaries should be understood to have meanings consistent with the contents of the relevant fields, and would not be interpreted overly idealized or overly formal unless clearly defined herein. As described in the present specification, a range of values is used as a shorthand to describe each and every numerical value in the range, and any number within that range may be chosen as the end-value of that range.

The present application provides a system for continuous cell production, comprising:

a culture container, and

a polymer blended layer arranged on the inner surface of the culture container,

wherein, the polymer blended layer is a pH-responsive polymer blended with nylon.

In one embodiment, system for continuous cell production further comprise a culture medium and/or a cell.

In one embodiment, the pH-responsive polymer is selected from the group consisting of polymers with amine groups, chitosan, polylysine, polyacrylamine, polyacrylamide, polyhexamethylenediamine, polypropylenediamine, polybutylenediamine, polypentamethylenediamine and combinations thereof.

In one embodiment, the ratio of the blended weight of the pH-responsive polymer and nylon is 1:1 to 10:1.

In one embodiment, the polymer blended layer is prepared by mixing solutions of the pH-responsive polymer and nylon uniformly, coating onto the inner surface of the container, and drying it. In another embodiment, the polymer blended layer is a coating layer of the pH-responsive polymer with a plurality of nylon regions.

Besides, the present application also provides a method for continuous cell production comprising:
(1) providing the system of the present application,
(2) controlling the pH value of the culture medium to the range of 6.9-7.4 and cultivating the cell, and
(3) adjusting the pH value of the culture medium to the range of 7.5-8.5 to detach the cells.

In one embodiment, Step (2) and Step (3) are performed repeatedly. Preferably, Step (2) and Step (3) may be performed repeatedly more than 3 times.

In one embodiment, the cell is selected from the group consisting of stem cell, preferably adipose-derived stem cell, mesenchymal stem cell or skin stem cell; fibroblast cell, preferably foreskin fibroblast or embryonic fibroblast; epithelial cell, preferably proximal renal tubular epithelial cell; cancer cell, preferably epithelial lung cancer cell; keratinocyte; corneal stromal cell and epidermal cell.

The following enumerates several embodiments as examples to illustrate the implementation of the present application. Those having ordinary skill in the art can easily understand the advantages and effects of the present invention through the content of the present specification, and make various modifications and changes without departing from the spirit of the present invention to implement or apply the content of the invention.

### Example 1—Preparation of the System for Continuous Cell Production

One aspect of the application is homogeneous blending. First, the pH-responsive polymer (chitosan as an example) and nylon (nylon-6,6) are dissolved in a co-solvent (formic acid or acetic acid as an example) at a weight ratio of 1:1 to 10:1 (take a weight ratio of 5:1 as an example), and the solution is stirred evenly at room temperature. Next, the prepared solution is poured into a TCPS dish, dried in a 60° C. oven 0/N, neutralized with 0.5N NaOH, rinsed with deionized water, sterilized under UV light for one hour, and then ready to use.

Another aspect is island-like blending, in which the solution of the pH-responsive polymer (chitosan as an example) is first coated on a TCPS dish and dried, and then the nylon solution is dripped onto the coating at different positions (The weight ratio of chitosan to nylon is 1:1 to 10:1). Multiple island-like nylon regions are formed on the surface, and it can be used after drying, neutralizing, rinsing and sterilization in the same way.

### Example 2—Experiment of Cells Cultured in Systems with Different Materials

In this experiment, five culture systems were used for continuous cell culture, respectively:
1. CS (AA): Acetic acid is used as a solvent, and chitosan is used alone to coat TCPS dishes.
2. CS (FA): Formic acid is used as a solvent, and chitosan is used alone to coat TCPS dishes.
3. PCL/CS: Acetic acid is used as a co-solvent, and polycaprolactone (PCL) and chitosan were blended homogeneously at a weight ratio of 1:5 and coated on TCPS dishes.
4. NL/CS: Formic acid is used as a co-solvent, and nylon and chitosan were blended homogeneously at a weight ratio of 1:5 and coated on TCPS dishes.
5. GLTN/CS: Acetic acid is used as a co-solvent, and gelatin and chitosan were blended homogeneously at a weight ratio of 1:5 and coated on TCPS dishes.

The Hs68 cell line is used in this example for continuous culture experiments. Cells were first seeded in the above culture systems using a pH 6.99 medium and allowed to attach and grow for 48 hours (expansion phase). Then the original medium is removed and a pH 7.65 medium is added and remained for 1 hour to allow the cells to complete detachment and harvest (harvest period). The above operation is one culture cycle. The steps, adding pH 6.99 culture medium for 48 hours and then adjusting to pH 7.65 for 1 hour to harvest cell, are repeated for 4 consecutive cycles.

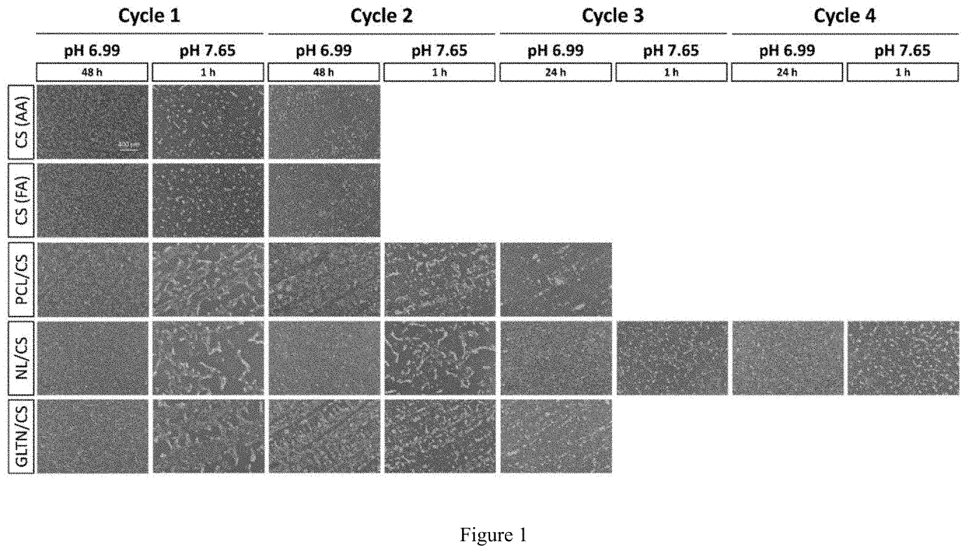

The experimental results are shown in FIG. 1, the continuous production of cells cannot be achieved by using chitosan (CS) alone. After the cells were detached in the first cycle, the cells cannot continue to grow and expand in the second cycle, regardless of the choice of solvent (acetic acid or formic acid). On PCL/CS and GLTN/CS, although the cells were detached when the pH value of the medium was increased in the first cycle, the extent of cell growth and expansion has been significantly reduced after continuing to culture for 48 hours in the second cycle. Cell growth and expansion gradually decrease with the increase in the number of cycles, and finally, present a sluggish state. In the third cycle, the cells have almost completely failed to keep growing. On the contrary, the effect of cell growth/expansion and cell detachment in each cycle can be clearly seen on NL/CS and can reach at least 3-4 cycles. It shows that pH-responsive polymer blended with nylon can be used for continuous cell production. Unlike the traditional cell culture method, which has to dilute and re-seed the cells for culture after the cells are harvested.

### Example 3—Analysis of Different Materials for Cell Culture

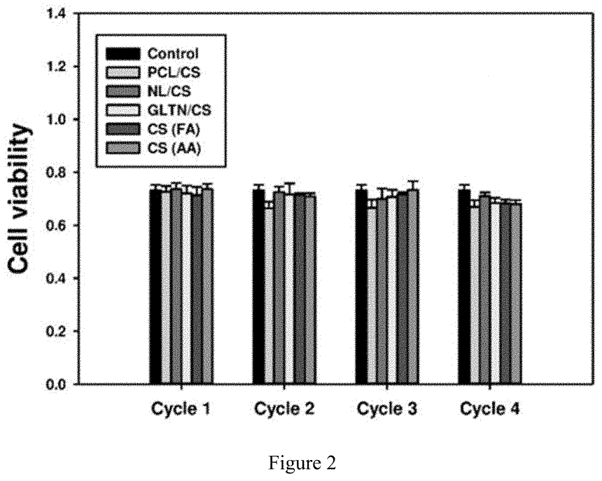

It is concerned that there may be material dissolution causing toxicity during repeated operations and rendering the cells of some blended material groups lead sluggish. In this experiment, therefore, the cell culture medium of each material cultured for 48 hours in the expansion phase in each cycle in Example 2 (extraction medium) is collected. The Hs68 cell line is used and seeded 3500 cells/well in a 96-well TCPS dish. The extraction mediums from each cycle above was added and the cells were cultured for 48 hours. The cells were collected, and the cell viability was analyzed by MTT (3-(4,5-dimethylthiazol-2-yl)-2,5-dephenyltetrazolium bromide) assay. It can be seen from FIG. 2 that this concern can be ruled out. The viability of the cells cultured in the extraction mediums from different materials in each cycle is equivalent, and there is no decrease in viability due to the toxicity of the materials that may dissolve.

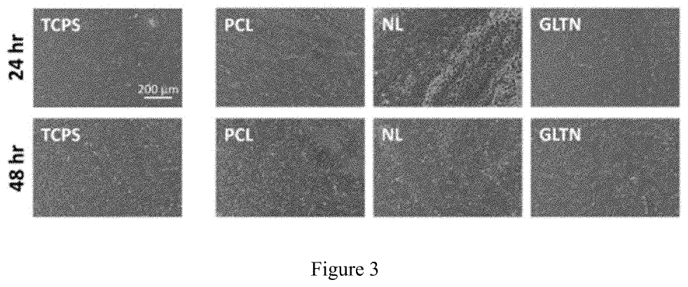

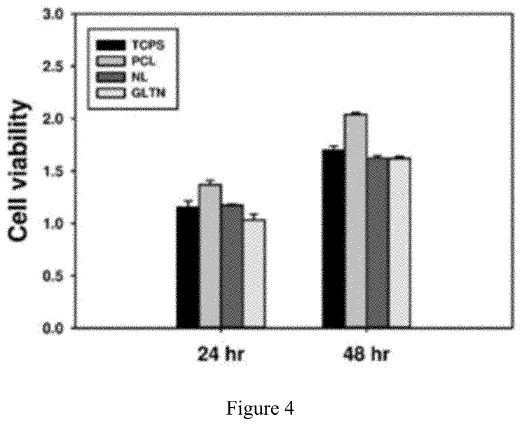

In addition, this experiment further explores the growth and viability of cells cultured on single materials. In this experiment, the bottoms of the TCPS dish were coated with single materials, polycaprolactone (PCL), nylon (NL) or gelatin (GLTN), and the Hs68 cell line was seeded. Cell growth was observed at 24 hours and 48 hours of culture, and the cell viability was analyzed by MTT assay. The results shown in FIG. 3, cells cultured on TCPS dishes coated with different single materials are comparable. The cells can attach well and grow to the full plate no matter on which material. At the same time, from the analysis results of the cell viability in FIG. 4, it can be seen that there is no significant difference in the cell viability of cells cultured on various materials, and even the viability of cells cultured on PCL is slightly better than other materials. However, the blend of PCL/CS has been proven that it is unable to continuously culture for multiple cycles in Example 2, which shows the reason that the blend of NL/CS can be used for continuous cell production is not simply the material-to-cell properties of nylon itself.

From this example, it can be known that the cells can complete continuous cell production on the NL/CS polymer blended layer is not because nylon can improve the viability of cultured cell, nor because other polymer materials would dissolve toxicity to reduce cell viability. It is because nylon and pH-responsive polymers are complementary to each other after blending. Although other polymer blended materials, such as PCL, used in prior arts are capable to detach cells, which cannot be cultured continuously for multiple cycles.

### Example 4—Experimental of Cell Culture with Different Cells and Blending Ways

In this experiment, Hs68 (fibroblasts), adipose-derived stem cells (ADSC), and human proximal tubular epithelial cells (hRPTEC) were cultured on a culture dish coated with homogeneous blended NL/CS (1:5). In addition, Hs68 (fibroblasts) and human proximal tubular epithelial cells (hRPTEC) were cultured on a culture dish coated with island-like blended NL/CS (1:5). The culture conditions and methods were the same as in Example 2.

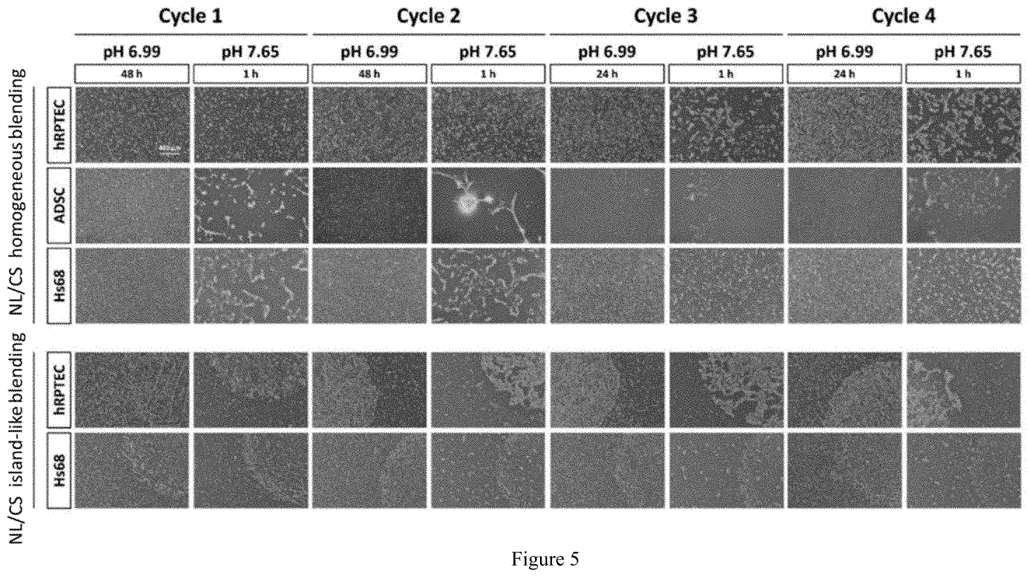

The experimental results are shown in FIG. 5. Good effects of growth/expansion and detachment can be achieved by adjusting the pH value of the medium no matter whether culturing Hs68, ADSC, or hRPTEC, or the system of homogeneous blending or island-like blending, and the cell production can be performed in a continuous manner for at least 4 cycles. In summary of the technology of the invention and experimental results disclosed above, the system and method for continuous cell production disclosed in the present application can continuously and periodically drive multiple cycles of cell production, providing very substantial help and benefits for academic research and industrial application.

What is claimed is:

1. A system for continuous cell production, comprising:
a culture container, and
a polymer blended layer arranged on the inner surface of the culture container,
wherein, the polymer blended layer is a pH-responsive polymer blended with nylon.

2. The system of claim 1, further comprising a culture medium and/or a cell.

3. The system of claim 1, wherein the pH-responsive polymer is selected from the group consisting of polymers with amine groups, chitosan, polylysine, polyacrylamine, polyacrylamide, polyhexamethylenediamine, polypropylenediamine, polybutylenediamine, polypentamethylenediamine and combinations thereof.

4. The system of claim 1, wherein the ratio of the blended weight of the pH-responsive polymer and nylon is 1:1 to 10:1.

5. The system of claim 1, wherein the polymer blended layer is prepared by mixing solutions of the pH-responsive polymer and nylon uniformly, coating onto the inner surface of the container, and drying it.

6. The system of claim 1, wherein the polymer blended layer is a coating layer of the pH-responsive polymer with a plurality of nylon regions.

7. A method for continuous cell production comprising:
(1) providing the system of claim 2,
(2) controlling the pH value of the culture medium to the range of 6.9-7.4 and cultivating the cell, and
(3) adjusting the pH value of the culture medium to the range of 7.5-8.5 to detach the cells.

8. The method of claim 7, wherein Step (2) and Step (3) are performed repeatedly.

9. The method of claim 8, wherein Step (2) and Step (3) may be performed repeatedly more than 3 times.

10. The system of claim 2, wherein the cell is selected from the group consisting of stem cell, preferably adipose-derived stem cell, mesenchymal stem cell or skin stem cell; fibroblast cell, preferably foreskin fibroblast or embryonic fibroblast; epithelial cell, preferably proximal renal tubular epithelial cell; cancer cell, preferably epithelial lung cancer cell; keratinocyte; corneal stromal cell and epidermal cell.
